(19) United States
(12) Patent Application Publication
(10) Pub. No.: US 2026/0321397 A1
(43) Pub. Date: Oct. 15, 2026

(54) METHOD AND SYSTEMS FOR AUTOMATED NAMING SERIES DESCRIPTIONS

(71) Applicant: GE Precision Healthcare LLC, Waukesha, WI (US)

(72) Inventors: Chelsey Lewis, Brookfield, WI (US); Masaru Seto, Hino (JP); Ayane Sato, Kokubunji-shi (JP)

(21) Appl. No.: 19/644,724

(22) Filed: Apr. 10, 2026

Related U.S. Application Data
(60) Provisional application No. 63/787,327, filed on Apr. 11, 2025.

(51) Int. Cl. G16H 30/20; G16H 40/67; G06F 3/04847

(52) U.S. Cl. CPC G16H 30/20; G16H 40/67; G06F 3/04847

(57) ABSTRACT

Various methods and systems are provided for generating automated naming conventions and assisting the user during the operation of an imaging system at a site. Via a management interface, a user may customize the scan and/or reconstruction series descriptions utilizing system parameters to customize the naming automatically rather than manually. During the modification of a protocol at protocol management, scan-time or post scan in accordance with the imaging system will then automatically update the series descriptions utilizing this automated naming.

### CROSS-REFERENCE TO RELATED APPLICATIONS

This application claims the benefit of and priority to U.S. Provisional Application No. 63/787,327, filed on Apr. 11, 2025, the disclosure of which is incorporated herein by reference in its entirety.

### BACKGROUND

Medical imaging typically utilizes protocols for a given suspected clinical use case. Within a protocol there typically contains either scan and/or image reconstructions both having series descriptions. Typically, the series descriptions that are transferred to the next user in the overall workflow from an imaging device by a technologist would be the Image Reconstruction Series description. Today systems utilize free text and thus there is a wide range across multiple vendors and sites how these series descriptions are displayed. Additionally with the rise of technologist shortages on the scan series and protocol naming standardization of the scan series naming could be standardized as well to bring harmonization and consistency from site to site. At both protocol management and scan time the technologists of these medical imaging systems may also make updates to parameters that are appropriate for the patient (e.g., a particular patient may require different protocols than are standard). However, the series description may not always match, and the technologist now must identify and update all descriptions manually.

### SUMMARY

This summary introduces concepts that are described in more detail in the detailed description. It should not be used to identify essential features of the claimed subject matter, nor to limit the scope of the claimed subject matter.

In an aspect of the described methods, an example method for naming images using an imaging system includes configuring, via a user interface, one or more parameters related to an imaging protocol in a specified output order, updating the imaging protocol based on the configured parameters, and providing a series description naming convention for the imaging protocol based on the specified output order of the parameters.

In another aspect of the described methods, another example method of naming images using an imaging system includes opening, via a user interface of the a workstation coupled to the imaging system, a scan protocol including a series description naming convention, wherein the series description naming convention is based on a specified output order of parameters set by a protocol manager, updating, a series description based on one or more parameters defined within the imaging protocol, detecting, via the workstation, whether a technologist has modified at least one parameter during setup or execution of an imaging scan, and in response to detecting the modification, re-executing the naming instructions to update the series description based on the modified parameter.

In yet another aspect of the described system, an example imaging apparatus includes a memory storing a protocol database including naming rules and one or more parameters associated with a scan prescription or a reconstruction task, and a processor configured to execute instructions stored in the memory to receive the one or more parameters, apply a user-defined parameter order to the one or more parameters, and generate a series description based on the user-defined parameter order.

### BRIEF DESCRIPTION OF DRAWINGS

Various aspects of this disclosure may be better understood upon reading the following detailed description and upon reference to the drawings in which:

FIG. 1 illustrates an exemplary imaging system 100 configured for CT imaging.

FIG. 2 illustrates an exemplary imaging system 200 similar to the imaging system 100 of FIG. 1.

FIG. 3 depicts an example method for automated naming of series descriptions.

FIG. 4 depicts an example user interface that may be presented to the user to configure the automatic naming.

FIG. 5 depicts an example method of updating naming or parameters at the time a scan is performed.

FIG. 6 depicts an example portion of the user interface that includes the updated series description.

FIG. 7 depicts an example interface that allows the user to enable smart series description in system preferences.

FIG. 8 depicts an example smart series description field that appears in the reconstruction job task if smart series description is enabled.

FIGS. 9A and 9B depicts the display of a result with smart series description enabled a result with smart series description with prefix enabled.

### DETAILED DESCRIPTION

Embodiments of the present disclosure will now be described, by way of example, with reference to the Figures, in which the description and embodiments of the subject matter disclosed herein relate to a standardization of naming for protocols for use during scans or imaging performed by an imaging system. The example imaging system illustrated herein includes a computed tomography (CT) imaging system. However, the subject patient positioning system and method may be used with any other imaging system, including but not limited to, a Photon Counting CT (PCCT) imaging system, an X-ray imaging system, a Magnetic Resonance Imaging (MRI) system, a Positron Emission Tomography (PET) imaging system, a Single-Photon Emission Computed Tomography (SPECT) imaging system, and combinations thereof (e.g., multi-modality imaging systems, such as PET/CT, PET/MR or SPECT/CT imaging systems). The present discussion of X-ray and CT imaging systems is provided merely as an example of a couple suitable imaging systems.

Within imaging the standardization of naming for protocols, series descriptions for both scan prescriptions (which may include both series and groups) and reconstructions, has a long overdue need to be filled. Today users can often find themselves in front of an imaging device deep inside changing parameters only to find they forgot to change the descriptions of their series and now on the PACs or post processing side there is a gap that will need to be rectified. In addition to the already existing hanging protocols, increasing use of AI results in a need to design and develop a standardized process for the imaging system that can uniquely determine a naming convention for the series description that is intelligently informed by the prescription parameters themselves with the allowance of customization. In some examples, this is an optional feature that may be turned on or off on a site-specific basis.

The following description relates to the embodiment of the system and method for enabling the automated naming convention within an associated imaging system. Traditionally the imaging systems, such as computed tomography (CT), would have an authorized user that manages protocols and series descriptions as one element of those protocols. This user largely takes on this task as manual activity that is currently not automated and is prone to user error. When a user makes a change to a prescription within a protocol it further can become an exacerbated issue if the imaging device allows for find and replace techniques to be utilized as the descriptions can further become difficult to adjust.

Additionally, during the operation of certain imaging modalities, such as computed tomography (CT) imaging, a scan technologist may make decisions relevant to the patient condition and thus change a parameter that affects the image parameters. For example, for a patient with metal (e.g., implants, pins, metallic dental fillings, etc.) this could look like enabling a metal artifact reduction feature. In a further example, this could be adding a reconstruction on demand for a particular lesion of interest that was not known prior to the scan. In both these cases having an automated naming convention that can either populate the field if turned on or populate an entire series description is largely beneficial.

Thus, the embodiment of the method and system of a database to store executable instructions for series description naming based on scan and/or reconstruction/image parameters respectively as shown and described herein. This example method reduces the error both at scan time and protocol management by executing the naming each time the parameter is updated.

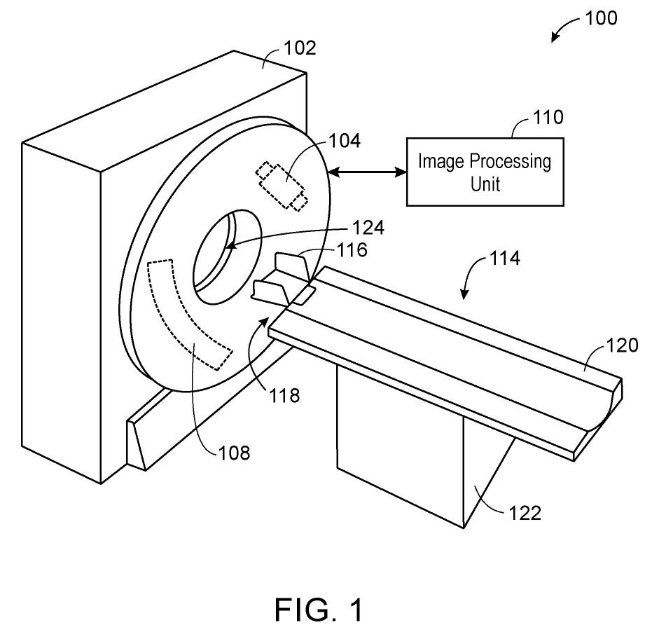

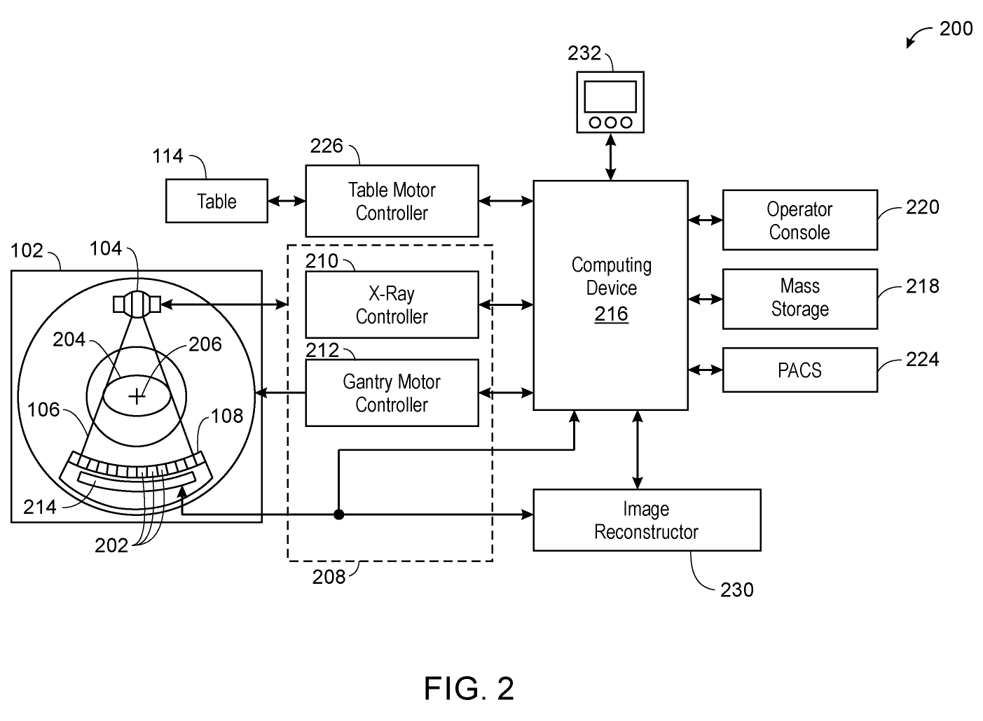

FIG. 1 illustrates an exemplary imaging system 100 configured for CT imaging. Particularly, the imaging system 100 is configured to image a subject such as a patient, an inanimate object, one or more manufactured parts, and/or foreign objects such as dental implants, stents, and/or contrast agents present within the body. In one embodiment, the imaging system 100 includes a gantry 102, which in turn, may further include at least one X-ray source 104 configured to project a beam of X-ray radiation 106 (see FIG. 2) for use in imaging the subject laying on and/or cantilevered off of an end of a table 114. Specifically, the X-ray source 104 is configured to project the X-ray radiation beams 106 towards a detector array 108 positioned on the opposite side of the gantry 102. Although FIG. 1 depicts a single X-ray source 104, in certain embodiments, multiple X-ray sources and detectors may be employed to project a plurality of X-ray radiation beams 106 for acquiring projection data at different energy levels corresponding to the patient. In some embodiments, the X-ray source 104 may enable dual-energy gemstone spectral imaging (GSI) by rapid peak kilovoltage (kVp) switching. In some embodiments, the X-ray detector employed is a photon-counting detector which is capable of differentiating X-ray photons of different energies. In other embodiments, two sets of X-ray sources and detectors are used to generate dual-energy projections, with one set at low-kVp and the other at high-kVp. It should thus be appreciated that the methods described herein may be implemented with single energy acquisition techniques as well as dual energy acquisition techniques.

In certain embodiments, the imaging system 100 further includes an image processor unit 110 configured to reconstruct images of a target volume of the subject using an iterative or analytic image reconstruction method. For example, the image processor unit 110 may use an analytic image reconstruction approach such as filtered back projection (FBP) to reconstruct images of a target volume of the patient. As another example, the image processor unit 110 may use an iterative image reconstruction approach such as advanced statistical iterative reconstruction (ASIR), conjugate gradient (CG), maximum likelihood expectation maximization (MLEM), model-based iterative reconstruction (MBIR), and so on to reconstruct images of a target volume of the subject. As described further herein, in some examples the image processor unit 110 may use both an analytic image reconstruction approach, such as FBP, in addition to an iterative image reconstruction approach.

In some CT imaging system configurations, an X-ray source projects a cone-shaped X-ray radiation beam which is collimated to lie within an X-Y-Z plane of a Cartesian coordinate system and generally referred to as an "imaging plane." The X-ray radiation beam passes through an object being imaged, such as the patient or subject. The X-ray radiation beam, after being attenuated by the object, impinges upon an array of detector elements. The intensity of the attenuated X-ray radiation beam received at the detector array is dependent upon the attenuation of a radiation beam by the object. Each detector element of the array produces a separate electrical signal that is a measurement of the X-ray beam attenuation at the detector location. The attenuation measurements from all the detector elements are acquired separately to produce a transmission profile.

In some CT systems, the X-ray source and the detector array are rotated with a gantry within the imaging plane and around the object to be imaged such that an angle at which the radiation beam intersects the object constantly changes. A group of X-ray radiation attenuation measurements, e.g., projection data, from the detector array at one gantry angle is referred to as a "view." A "scan" of the object includes a set of views made at different gantry angles, or view angles, during one revolution of the X-ray source and detector. It is contemplated that the benefits of the methods described herein accrue to medical imaging modalities other than CT, so as used herein the term "view" is not limited to the use as described above with respect to projection data from one gantry angle. The term "view" is used to mean one data acquisition whenever there are multiple data acquisitions from different angles, whether from a CT, positron emission tomography (PET), or single-photon emission CT (SPECT) acquisition, and/or any other modality including modalities yet to be developed as well as combinations thereof in fused embodiments.

The projection data is processed to reconstruct an image that corresponds to a two-dimensional slice taken through the object or, in some examples where the projection data includes multiple views or scans, a three-dimensional rendering of the object. One method for reconstructing an image from a set of projection data is referred to in the art as the filtered back projection technique. Transmission and emission tomography reconstruction techniques also include statistical iterative methods such as maximum likelihood expectation maximization (MLEM) and ordered-subsets expectation-reconstruction techniques as well as iterative reconstruction techniques. This process converts the attenuation measurements from a scan into integers called "CT numbers" or "Hounsfield units," which are used to control the brightness of a corresponding pixel on a display device.

To reduce the total scan time, a "helical" scan may be performed. To perform a "helical" scan, the patient is moved while the data for the prescribed number of slices is acquired. Such a system generates a single helix from a cone beam helical scan. The helix mapped out by the cone beam yields projection data from which images in each prescribed slice may be reconstructed.

As used herein, the phrase "reconstructing an image" is not intended to exclude embodiments of the present invention in which data representing an image is generated but a viewable image is not. Therefore, as used herein, the term "image" broadly refers to both viewable images and data representing a viewable image. However, many embodiments generate (or are configured to generate) at least one viewable image.

FIG. 2 illustrates an exemplary imaging system 200 similar to the imaging system 100 of FIG. 1. In accordance with aspects of the present disclosure, the imaging system 200 is configured for imaging a subject 204. In one embodiment, the imaging system 200 includes the detector array 108 (see FIG. 1). The detector array 108 further includes a plurality of detector elements 202 that together sense the X-ray radiation beam 106 (see FIG. 2) that pass through the subject 204 (such as a patient) to acquire corresponding projection data. Accordingly, in one embodiment, the detector array 108 is fabricated in a multi-slice configuration including the plurality of rows of cells or detector elements 202. In such a configuration, one or more additional rows of the detector elements 202 are arranged in a parallel configuration for acquiring the projection data.

In certain embodiments, the imaging system 200 is configured to traverse different angular positions around the subject 204 for acquiring desired projection data. Accordingly, the gantry 102 and the components mounted thereon may be configured to rotate about a center of rotation 206 for acquiring the projection data, for example, at different energy levels. Alternatively, in embodiments where a projection angle relative to the subject 204 varies as a function of time, the mounted components may be configured to move along a general curve rather than along a segment of a circle.

As the X-ray source 104 and the detector array 108 rotate, the detector array 108 collects data of the attenuated X-ray beams. The data collected by the detector array 108 undergoes pre-processing and calibration to condition the data to represent the line integrals of the attenuation coefficients of the scanned subject 204. The processed data are commonly called projections.

In some examples, the individual detectors or detector elements 202 of the detector array 108 may include photon-counting detectors which register the interactions of individual photons into one or more energy bins. It should be appreciated that the methods described herein may also be implemented with energy-integrating detectors.

The acquired sets of projection data may be used for basis material decomposition (BMD). During BMD, the measured projections are converted to a set of material-density projections. The material-density projections may be reconstructed to form a pair or a set of material-density map or image of each respective basis material, such as bone, soft tissue, and/or contrast agent maps. The density maps or images may be, in turn, associated to form a volume rendering of the basis material, for example, bone, soft tissue, and/or contrast agent, in the imaged volume.

Once reconstructed, the basis material image produced by the imaging system 200 reveals internal features of the subject 204, expressed in the densities of two basis materials. The density image may be displayed to show these features. In traditional approaches to diagnosis of medical conditions, such as disease states, and more generally of medical events, a radiologist or physician would consider a hard copy or display of the density image to discern characteristic features of interest. Such features might include lesions, sizes and shapes of particular anatomies or organs, and other features that would be discernable in the image based upon the skill and knowledge of the individual practitioner.

In one embodiment, the imaging system 200 includes a control mechanism 208 to control movement of the components such as rotation of the gantry 102 and the operation of the X-ray source 104. In certain embodiments, the control mechanism 208 further includes an X-ray controller 210 configured to provide power and timing signals to the X-ray source 104. Additionally, the control mechanism 208 includes a gantry motor controller 212 configured to control a rotational speed and/or position of the gantry 102 based on imaging requirements.

In certain embodiments, the control mechanism 208 further includes a data acquisition system (DAS) 214 configured to sample analog data received from the detector elements 202 and convert the analog data to digital signals for subsequent processing. The DAS 214 may be further configured to selectively aggregate analog data from a subset of the detector elements 202 into so-called macro-detectors, as described further herein. The data sampled and digitized by the DAS 214 is transmitted to a computer or computing device 216. In one example, the computing device 216 stores the data in a storage device or mass storage 218. The storage device 218, for example, may include a hard disk drive, a floppy disk drive, a compact disk-read/write (CD-R/W) drive, a Digital Versatile Disc (DVD) drive, a flash drive, and/or a solid-state storage drive.

Additionally, the computing device 216 provides commands and parameters to one or more of the DAS 214, the X-ray controller 210, and the gantry motor controller 212 for controlling system operations such as data acquisition and/or processing. In certain embodiments, the computing device 216 controls system operations based on operator input. The computing device 216 receives the operator input, for example, including commands and/or scanning parameters via an operator console 220 operatively coupled to the computing device 216. The operator console 220 may include a keyboard (not shown) or a touchscreen to allow the operator to specify the commands and/or scanning parameters.

Although FIG. 2 illustrates one operator console 220, more than one operator console may be coupled to the imaging system 200, for example, for inputting or outputting system parameters, requesting examinations, plotting data, and/or viewing images. Further, in certain embodiments, the imaging system 200 may be coupled to multiple displays, printers, workstations, and/or similar devices located either locally or remotely, for example, within an institution or hospital, or in an entirely different location via one or more configurable wired and/or wireless networks such as the Internet and/or virtual private networks, wireless telephone networks, wireless local area networks, wired local area networks, wireless wide area networks, wired wide area networks, etc.

In one embodiment, for example, the imaging system 200 either includes, or is coupled to, a picture archiving and communications system (PACS) 224. In an exemplary implementation, the PACS 224 is further coupled to a remote system such as a radiology department information system, hospital information system, and/or to an internal or external network (not shown) to allow operators at different locations to supply commands and parameters and/or gain access to the image data.

The computing device 216 uses the operator-supplied and/or system-defined commands and parameters to operate a table motor controller 226, which in turn, may control a table 114 which may be a motorized table. Specifically, the table motor controller 226 may move the table 114 for appropriately positioning the subject 204 in the gantry 102 for acquiring projection data corresponding to the target volume of the subject 204.

As previously noted, the DAS 214 samples and digitizes the projection data acquired by the detector elements 202. Subsequently, an image reconstructor 230 uses the sampled and digitized X-ray data to perform high-speed reconstruction. Although FIG. 2 illustrates the image reconstructor 230 as a separate entity, in certain embodiments, the image reconstructor 230 may form part of the computing device 216. Alternatively, the image reconstructor 230 may be absent from the imaging system 200 and instead the computing device 216 may perform one or more functions of the image reconstructor 230. Moreover, the image reconstructor 230 may be located locally or remotely, and may be operatively connected to the imaging system 200 using a wired or wireless network. Particularly, one exemplary embodiment may use computing resources in a "cloud" network cluster for the image reconstructor 230.

In one embodiment, the image reconstructor 230 stores the images reconstructed in the storage device 218. Alternatively, the image reconstructor 230 may transmit the reconstructed images to the computing device 216 for generating useful patient information for diagnosis and evaluation. In certain embodiments, the computing device 216 may transmit the reconstructed images and/or the patient information to a display or display device 232 communicatively coupled to the computing device 216 and/or the image reconstructor 230. In some embodiments, the reconstructed images may be transmitted from the computing device 216 or the image reconstructor 230 to the storage device 218 for short-term or long-term storage.

Though a CT system is described by way of example, it should be understood that the present technology may also be used on other imaging modalities, such as X-ray imaging systems, magnetic resonance imaging (MRI) systems, nuclear medicine imaging systems, positron emission tomography (PET) imaging systems, single-photon emission computed tomography (SPECT) imaging systems, ultrasound imaging systems, and combinations thereof (e.g., multi-modality imaging systems, such as PET/CT or PET/MR imaging systems). The present discussion of a CT imaging modality is provided merely as an example of one suitable imaging modality.

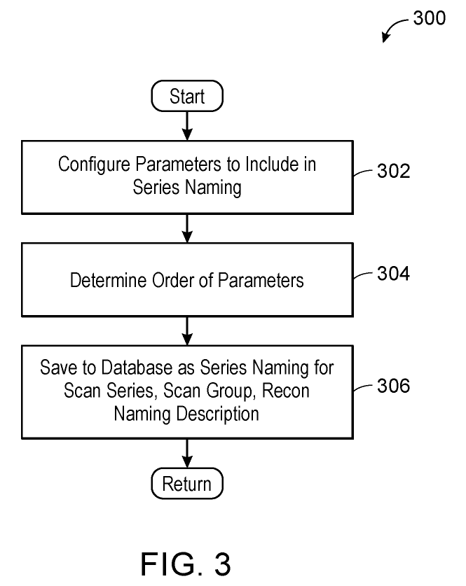

FIG. 3 depicts an example method 300 for setup of automated naming of series descriptions. The example method 300 may be performed, in some examples, by a processor associated with a workstation remotely coupled to the example imaging system 100 and/or an operator console 220 of the imaging system. An example method 300 depicted in FIG. 3 begins by configuring parameters to include series naming or description (block 302). The series description may include a Scan Series Description or a Reconstruction Series Description (e.g., an Image Series Description). In some examples, these parameters may be derived from scan prescriptions parameters, reconstruction settings, user-defined fields, or system-derived values. Example scan prescription parameters may include tube voltage (kVp), tube current (mA), pitch, collimation, gantry rotation time, acquisition mode (e.g., axial, helical, volumetric), detector configurations, patient position or orientation (e.g., supine, prone, head first, feet first). In some examples, reconstruction parameters may include reconstruction type (e.g., axial, coronal, sagittal), reconstruction kernel (e.g., bone, soft tissue, etc.), slice thickness, field of view (FOV), iterative reconstruction level, (e.g., ASIR, MBIR), use of metal artifact reduction (MAR), or dual energy/spectral settings. User-defined fields may include group names (e.g., spine), user-entered labels, or optional prefixes and suffixes. In some examples, the system-derived parameters may include detected presence of metal artifacts, automatically generated reconstruction variants, and additional reconstructions triggered by technologist modification. Thus, at block 302, the method 300 determines which of the parameters will be included in the automated naming of the series description. In some examples, the user configures the parameters by selecting parameters from a pre-defined list. In some examples, the system provides suggestions of parameters (e.g., as a starting point) in addition to allowing the user to select from a pre-defined list. It should be understood that these parameters may be example parameters associated with a certain type of imaging system (e.g., a CT imaging system) and are merely examples. Other types of imaging systems (e.g., MRI, X-ray imaging, and combined PET/CT, PET/MR, SPECT/CT, or SPECT/MR systems) may include different and/or additional parameters.

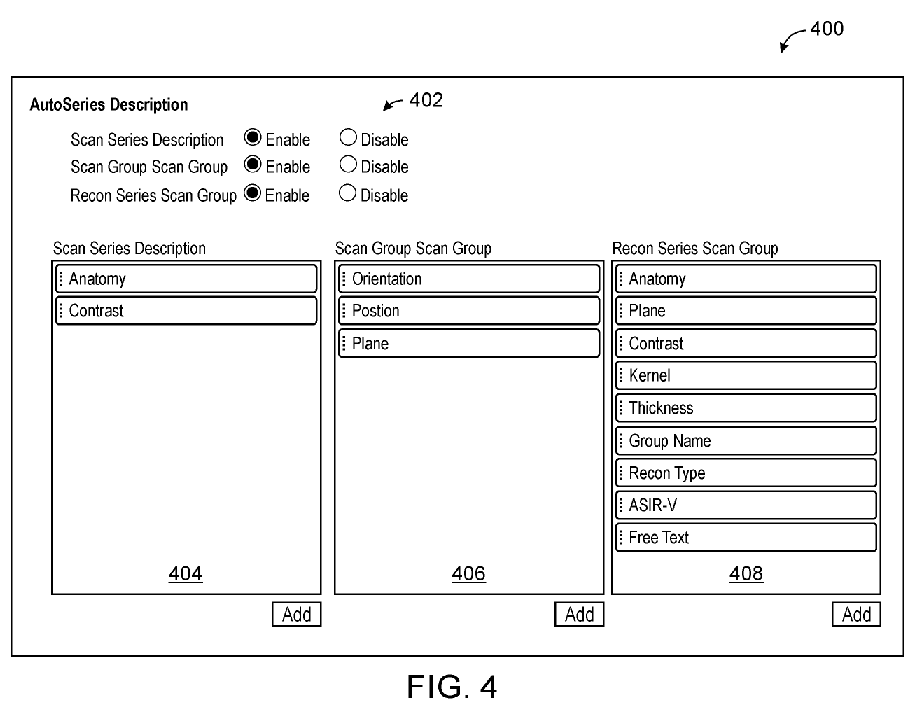

The method 300 continues with determining an order of parameters (block 304). For example, the user can customize the list so that the parameters are displayed in a preferred order, such as an order depicted in FIG. 4. For example, the user can select which of the existing parameters to include first, and/or add additional parameters. The user can customize the parameters included in the description of each of the scan series, scan group, and reconstruction series scan group. For example, a user may prefer the scanned anatomy to be displayed in the description prior to the contrast value for a scan series. After the user has customized the description order, the method then continues with saving the changes to a database (e.g., a protocol database) accessible to the imaging console and/or a remote workstation (block 306). Once the order is saved, the system will automatically apply this order to the naming descriptions.

FIG. 4 depicts an example user interface 400 that may be presented to the user (e.g., via a remote workstation, a display 232, or the operator console 220) to configure the automatic naming. The example user interface 400 allows the user to enable or disable automatic naming using a series of toggles 402. The user is able to independently choose whether or not to allow automatic naming for the scan series description, the scan group, and the reconstruction series scan group. In the illustrated example, the user interface includes selectable radio buttons, but any other option to toggle the automatic naming on and off may be used instead, including a checkbox, toggle/switch, etc. If the automatic description is enabled for one or more category, the user interface may allow the user to add, reorder, or remove parameters in each description or group using one of a plurality of sections 404, 406, 408. In the illustrated example, the sections 404, 406, 408 for reordering the parameters of all categories appear on the same view of the user interface as a billable filed box under the category description. Alternatively, the sections 404, 406, 408 may be displayed on separate user interface views and/or may be displayed in a different manner. In some examples, the user may add parameters into the section by using an add button and selecting parameters from a pre-populated or pre-specified list that may pop-up. Alternatively, the user may be able to add parameters in a different manner. After parameters are added, the user may be able to reorder by dragging and dropping the different parameters within the section. Alternatively, the user interface 400 may use another manner of re-ordering parameters, such as using up and down arrows to move the selected parameter up or down in the order of the displayed list. Other ordering or ranking systems may also be used. In some examples, the user can order the naming convention using a free text format. For example, if the naming is configured as “<GroupName>: <ReconType>, <ImageThickness>mm” by the user, then the series description is automatically updated to be “Spine: Snd, 0.625mm” when saved for a specific example protocol. The <parameter> may come from scan/reconstruction prescription.

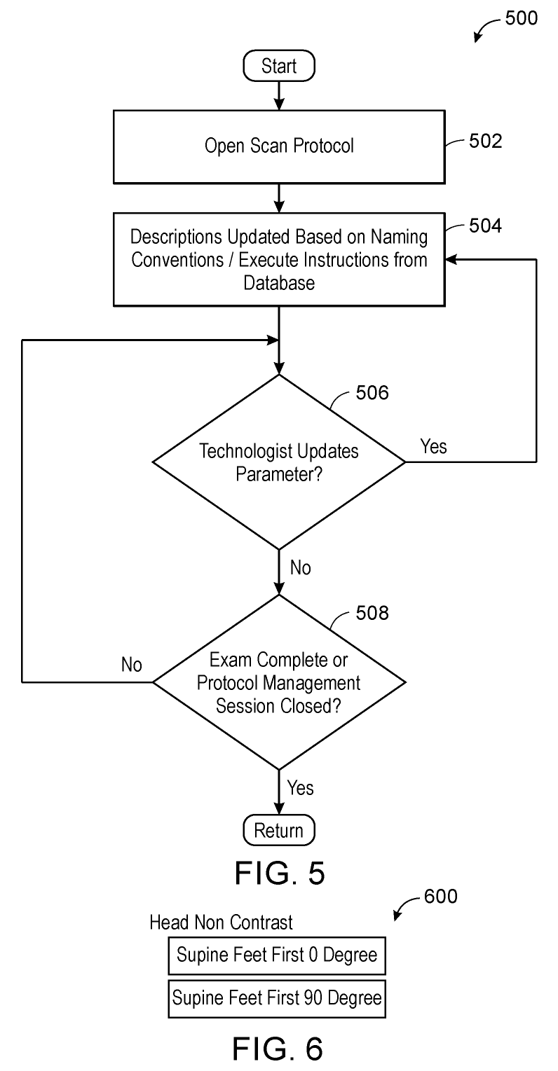

FIG. 5 depicts an example method 500 of updating naming settings or parameters at the time a scan is performed. The example method 500 may be performed by, in some examples, by a processor associated with a workstation remotely coupled to the example imaging system 100 and/or an operator console 220 of the imaging system. The method 500 beings by opening an existing protocol, which may have been created by a protocol manager or technician (block 502). The example protocol may include the naming settings created using the example method 300 of FIG. 3 and/or the user interface 400 of FIG. 4. For example, the protocol may include one or more series descriptions, naming templates and ordered parameters used for automatic naming.

The method 500 continues by updating the protocol based on naming conventions, which may include executing instructions from the database (e.g., the protocol database) (block 504). In some examples, the protocols may require updates due to changes by the protocol manager at a workstation remote from the imaging device. The method 500 continues by determining whether any updates or modifications have been made by the technologist at the imaging device (i.e., at the time the scan is performed, immediately prior to or following the scan) (block 506). If the technologist made changes, the method 500 returns to the updating step 504 (e.g., to re-execute the naming instructions or conventions), which may include saving the changes to the database. If the technologist didn’t make changes, the method 500 continues to determine if the exam is complete, or the protocol management session is closed (block 508). If not, the method 500 will continue to monitor for changes made by the technologist. If the session is complete or closed, the method 500 is complete. Afte the scan is complete, the imaging system will automatically apply the description naming convention that is associated with the protocol to the scan files and reconstruction results. Thus, any imaging scan run using the particular scan protocol selected will be named using the same naming convention associated with that protocol, thereby standardizing the naming for the protocol.

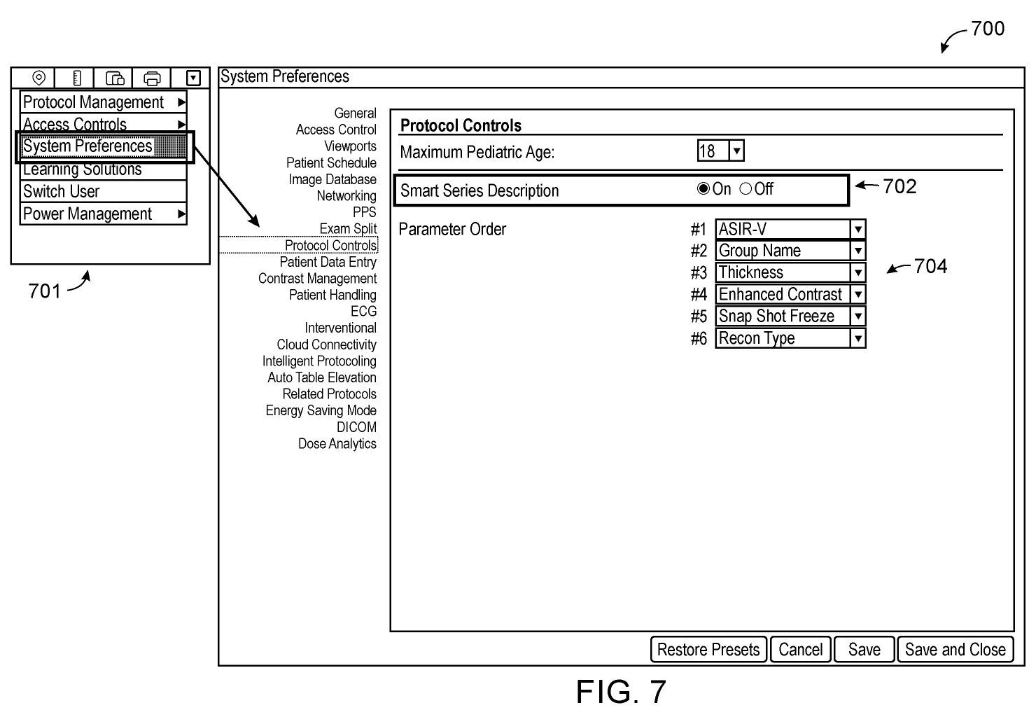

FIG. 6 depicts an example portion of the user interface 600 that includes the updated series description (e.g., Head Non-Contrast) and group description(s) (e.g., Supine Feet First 0 degree, Supine Feet First 90 degree). In some examples, this portion of the user interface 600 depicting the changes to the description may be a pop-up alert that is provided to the technician and/or protocol manager. For example, a pop-up alert may be presented to the user during block 504 of the example method of FIG. 5, indicating what updates were made (e.g., by a protocol manager) from the last time the system (e.g., that specific imaging system) ran the selected protocol. Additionally or alternatively, the pop-up alert may be provided if the technologist changes parameters in block 506. In such examples, the pop-up alert may be presented to the technologist to confirm the changes are accurate and/or desired, and/or may be presented to a protocol manager as an alert that a technologist changed the naming convention. In some examples, multiple series and group descriptions may be included. That is, any changes to the naming convention for the selected protocol may be indicated.

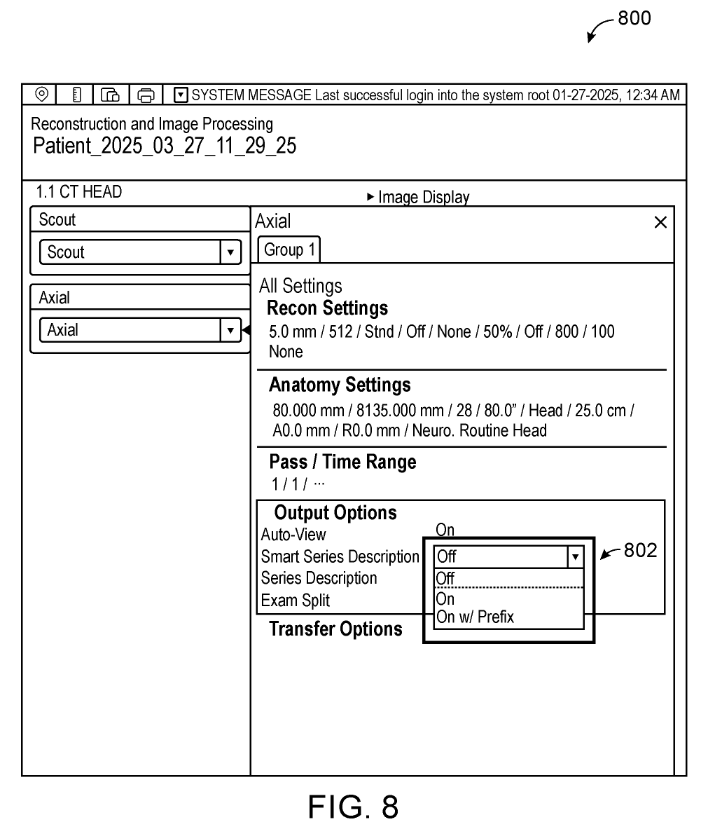

FIG. 7 depicts an example interface 700 that allows the user to enable smart series description in system preferences using a toggle 702. The interface may be accessed by selecting system preferences 701 from a menu of the interface 700. The user can select the parameter(s) to include in the series description in their preferred order using a selection menu in a parameter ordering region 704. The parameter order field will appear and/or be editable when the smart series description is enabled. The interface 700 provides centralized control over how automated naming rules are applied across scan prescriptions and reconstruction tasks. When accessed by an authorized user, such as a protocol manager or senior technologist, the interface presents a global toggle that determines whether the automated naming engine is active for the current imaging device or for all devices connected to a shared protocol management service.

In some embodiments, the interface 700 includes a control element such as a drop-down, checkbox, switch, or slider that allows the user to enable smart series description functionality. Upon enabling this feature, additional configuration options become available. These options may include a parameter selection panel listing all available parameters that may be included in an auto-generated naming string, such as slice thickness, reconstruction kernel, acquisition orientation, reconstruction type, metal artifact reduction status, or spectral imaging mode. Users may select or deselect individual parameters to tailor naming behavior to local workflow requirements.

The interface 700 may further include a parameter ordering region 704 that appears dynamically when the smart series description is activated. This region 704 enables users to specify the priority and sequence of parameters that will be incorporated into the final naming string. While the example interface 700 depicts the parameters in an ordered list with drop-down menus associated with each position on the list, other interfaces may include other ways for ordering or re-arranging the parameters. For example, in a drag-and-drop embodiment, each parameter is represented as a moveable element within the list, allowing users to reorder parameters visually and intuitively. When the order is modified, the system updates the stored configuration in real time or upon confirmation by the user.

In certain embodiments, the interface 700 includes a preview pane that displays a sample output showing how series descriptions will appear when the configured rules are applied to a representative set of parameters. This preview helps users validate the clarity, readability, and consistency of the naming format before committing changes system-wide.

Additional embodiments of FIG. 7 may include administrative controls such as permission restrictions, audit logs of parameter changes, or warnings if selected parameters create conflicting or ambiguous naming strings. The system may prevent saving configurations that produce invalid formats, exceed naming length limitations, or violate DICOM character constraints.

Through interface 700, users can establish consistent, site-wide rules for automated series description generation, ensuring that naming conventions remain standardized, reproducible, and synchronized across protocol management tools, imaging consoles, and downstream systems such as PACS or post-processing workstations.

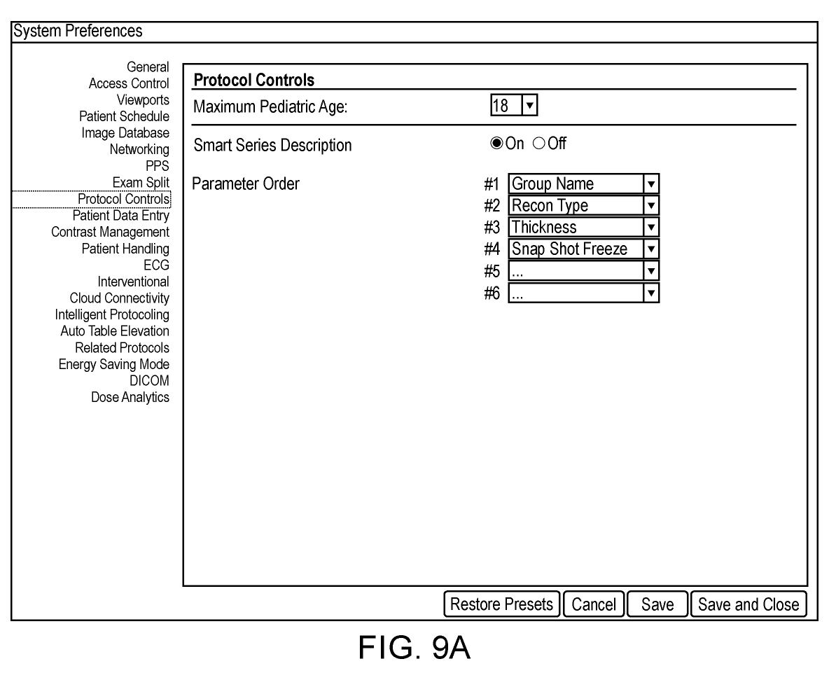

FIG. 8 depicts an example interface 800 that includes smart series description field 802 that appears in the reconstruction job task if smart series description is enabled. The user can select a reconstruction job task to apply smart series description to, and can select how to apply this feature in a review session. The user can turn this feature on or off. In some examples, the user can enable the feature with an additional prefix, which may be automatically manually entered. As shown, the interface integrates the automated naming engine directly into the reconstruction workflow, allowing the system to dynamically generate, preview, and apply series descriptions based on the reconstruction parameters selected for a given scan.

When a user initiates a reconstruction job—such as selecting an axial reconstruction, coronal MPR, or additional reconstructions—the interface 800 displays a designated field showing the series description that will be applied to the resulting image series. The system automatically populates this field based on the parameters associated with the selected reconstruction and the naming configuration defined during protocol management.

In one embodiment, the smart series description field 800 updates automatically whenever the user modifies a reconstruction parameter that affects naming, such as slice thickness, reconstruction kernel, or reconstruction type. As changes are made, the system evaluates the updated parameters and refreshes the description shown in the field. This allows the user to see the expected naming output before the reconstruction job is submitted.

The interface may allow the user to manually edit the text in the field if site settings permit manual overrides. In this embodiment, the system simply replaces the automatically generated text with the user‑entered text. If manual editing is not allowed, the field remains read‑only and reflects only the automated naming result.

In certain implementations, when a site has enabled optional prefix behavior, the prefix (if applicable) is displayed directly within the smart series description field. The user may enter, remove, or adjust the prefix before finalizing the reconstruction task, depending on administrative settings. The prefix is then combined with the automatically generated series description and displayed as a single text string.

Once the user confirms the reconstruction settings, the value shown in the smart series description field is stored as the final description for that reconstruction series. This ensures that the naming used in the reconstruction task is consistent with both site‑configured naming rules and any real‑time adjustments made at scan time.

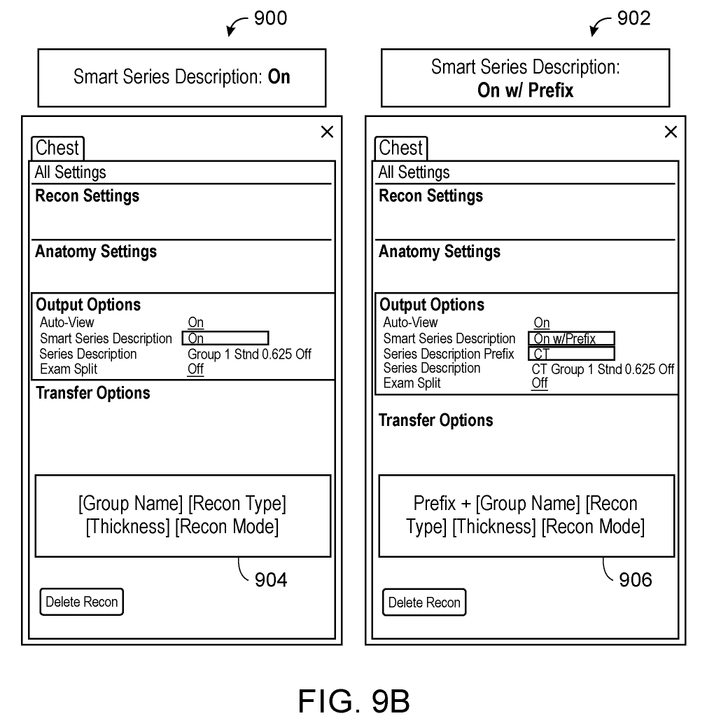

FIGS. 9A and 9B display an example interface 900 including a result 904with smart series description enabled and an example interface 902 including a result 906 with smart series description with prefix enabled. When the prefix is selected, the user has the option to add their own prefix before the auto-generated series description. As shown, the display reflects the naming string generated after the system evaluates the scan and reconstruction parameters associated with the selected reconstruction job. The naming engine applies the parameter ordering and naming configuration previously defined during protocol management and automatically produces a consistent, parameter-driven series description.

In this embodiment, the interface 900, 902 presents the resulting series description exactly as it will be stored for the reconstructed image series and subsequently transmitted to downstream systems such as PACS or post-processing workstations. The naming string may incorporate multiple parameter elements, including reconstruction type, slice thickness, kernel selection, or any other parameters designated for inclusion via the smart naming configuration. There may be an allowance for parameter list by scan type for example spectral reconstruction vs single energy. By displaying the description in the same location where manually entered text would typically appear, the system ensures a seamless integration between automated and conventional workflows.

If a site has enabled the prefix option, the prefix appears as part of the same naming field shown in FIGS. 9A and 9B. The prefix may be defined at the system preferences level or entered by the user at scan time, and it is merged directly with the automatically generated naming content to form a single, continuous description string. This allows the system to support site-specific naming patterns—such as department identifiers, priority codes, or institutional prefixes—while still maintaining consistency in the automated naming process.

The combined output shown in FIGS. 9A and 9B reflects the final naming result after all applicable rules, parameters, and optional modifications have been applied. Once the reconstruction task is completed, this naming string is stored with the reconstruction metadata and becomes the definitive series description for that dataset. By presenting the completed name directly within the results display, the system provides immediate confirmation to the user that the naming logic has been applied correctly, reducing the likelihood of errors and decreasing the need for manual renaming of series after image acquisition.

In some implementations, if the user adjusts reconstruction parameters or modifies a prefix prior to finalizing the reconstruction task, the description shown in FIGS. 9A and 9B updates automatically to reflect the latest parameter values. This ensures that the displayed naming string always corresponds to the configuration used for the actual reconstruction.

In an additional embodiment of the method and system of a database to store executable instructions for series description naming based on scan and/or recon/image parameters respectively. The naming is configured as a parameter directly within the field for the description. The system thereby stores these instructions from protocol management and then executes when this item is executed or run.

Embodiments of the present disclosure shown in the drawings and described above are example embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present invention. That is, features of the described embodiments can be combined with any appropriate aspect described above and optional features of any one aspect can be combined with any other appropriate aspect. Similarly, features set forth in dependent claims can be combined with non-mutually exclusive features of other dependent claims, particularly where the dependent claims depend on the same independent claim. Single claim dependencies may have been used as practice in some jurisdictions require them, but this should not be taken to mean that the features in the dependent claims are mutually exclusive.

What is claimed is:

1. A method for naming images using an imaging system, comprising:
configuring, via a user interface, one or more parameters related to an imaging protocol in a specified output order;
updating the imaging protocol based on the configured parameters; and
providing a series description naming convention for the imaging protocol based on the specified output order of the parameters.

2. The method of claim 1, where in a parameter of the one or more parameters may include a free text parameter.

3. The method of claim 1, wherein a parameter of the one or more parameters includes a scan parameter.

4. The method of claim 1, wherein a parameter of the one or more parameters includes a reconstruction parameter.

5. The method of claim 1, where in the series description is a Scan Series Description.

6. The method of claim 1, where in the series description is a Reconstruction Series Description.

7. The method of claim 1, wherein the series description is an Image Series Description.

8. The method of claim 1, wherein the one or more parameters are configured in the specified output order via a protocol manager of an operator workstation remotely coupled to the imaging system.

9. A method naming images using an imaging system, comprising:
opening, via a user interface of a workstation coupled to the imaging system, a scan protocol including a series description naming convention, wherein the series description naming convention is based on a specified output order of parameters set by a protocol manager;
updating, a series description based on one or more parameters defined within the imaging protocol;
detecting, via the workstation, whether a technologist has modified at least one parameter during setup or execution of an imaging scan; and
in response to detecting the modification, re-executing the naming convention to update the series description based on the modified parameter.

10. The method of claim 9, where in a parameter of the one or more parameters may include a free text parameter.

11. The method of claim 9, wherein a parameter of the one or more parameters includes a scan parameter.

12. The method of claim 9, wherein a parameter of the one or more parameters includes a reconstruction parameter.

13. The method of claim 9, further comprising continuing to monitor for modifications of at least one parameter during setup or execution of an imaging scan until the scan is complete.

14. The method of claim 9, further comprising continuing to monitor for modifications of at least one parameter during setup or execution of an imaging scan until a protocol management session is closed.

15. The method of claim 14, further comprising finalizing the updated series description upon completion of the example or closure of the protocol management session.

16. An imaging apparatus, comprising:
a memory storing a protocol database including naming rules and one or more parameters associated with a scan prescription or a reconstruction task; and
a processor configured to execute instructions stored in the memory to:
receive the one or more parameters;
apply a user-defined parameter order to the one or more parameters; and
generate a series description based on the user-defined parameter order.

17. The apparatus of claim 16, where in the series description is a Scan Series Description.

18. The apparatus of claim 16, where in the series description is a Reconstruction Series Description.

19. The apparatus of claim 16, wherein the series description is an Image Series Description.

20. The apparatus of claim 16, wherein the one or more parameters are configured in the specified output order via a protocol manager of an operator workstation remotely coupled to the imaging apparatus.
